(19) United States
(12) Patent Application Publication
(10) Pub. No.: US 2024/0230498 A1
(43) Pub. Date: Jul. 11, 2024

(54) Device and method for determining the physical properties of soils and particulate materials, particularly their static compaction

(71) Applicant: István SCHUBERT, Érd (HU)

(72) Inventor: István SCHUBERT, Érd (HU)

(21) Appl. No.: 18/406,953

(22) Filed: Jan. 8, 2024

(30) Foreign Application Priority Data
Jan. 9, 2023 (HU) P2300018

(51) Int. Cl. G01N 3/36; G01N 1/28; G01N 3/06; G01N 33/24

(52) U.S. Cl. CPC G01N 3/36; G01N 1/286; G01N 3/068; G01N 33/24

(57) ABSTRACT

Device as well as method for using the device for the determination of the physical properties of soils and particulate materials, particularly their static compaction.

The subject of the invention relates to a device for the determination of the physical properties of soils and particulate materials, particularly their static compaction, which contains a device housing, a load-transfer apparatus, a reference piece and measuring head, there is a seating for accommodating a part of the reference piece, where the seating is connected to the free opening positioned on the delimiting surface of the device housing, the load-transfer apparatus has a hydraulic slave cylinder and piston that has a medium-transfer pipe end, and the measuring head is located in the seating.

The subject of the invention also relates to a method for determining the properties of soils and particulate materials, particularly their static compaction, during which a device with a device housing, a measuring head connected to this, and a reference piece linked to the measuring head is used for the determination of static compaction, the device housing is placed on the surface of the soil or the particulate material to be tested, the device housing is placed under a load mass known of in itself and having a loading weight greater than 10 tonnes, then a load-transfer apparatus is inserted between the device housing and the load mass, following this the load-transfer apparatus is tensioned, and so by routing a part of the weight of the load mass through the device housing to the surface of the tested soil or particulate material load pressure is created, and a surface pressed by the load pressure is created, then after maintaining the given pressure for a resting period, a compacted layer is created, after the resting period has elapsed the load-transfer apparatus is tensioned more to create increased load pressure, which is routed to the compacted layer, then after maintaining the given increased pressure for a resting period, an increased pressure compacted layer is created, the pressure-increasing step is repeated at least four times with increasing load pressure, and so a complete loading cycle is performed, at the end of the complete loading cycle the measuring head is used to determine the value of the distance between the measuring head and the reference piece, then the load pressure is removed, and then the physical properties of the tested soil or particulate material are determined from the value obtained.

In the course of the construction of engineering facilities it is of fundamental importance to determine the physical properties of the soil or particulate material bearing the engineering facility. Including and particularly its static compaction. Various disc soil compaction measuring instruments have been developed for measuring these physical properties. Such devices are disclosed in the description of the ASTM standard ASTM-D1195-D1195M-2021, as well as in the device presentations accessible via the following Internet addresses:
www.youtube.com/watch?v=cC75pGoaIhQ&t=87s;
www.youtube.com/watch?v=FgZJQumBI28,
www.youtube.com/watch?v=am2D86yUK24&t=22s;
www.youtube.com/watch?v=BzryIVHC-ew

A general characteristic of the known solutions is that the depression caused by the load in the soil located under the base plate is determined using a mechanical or digital gauge fitted to the top of the base plate. However, the disadvantage of the devices is that, especially at the connection of the measuring gauge and the base plate, they consist of a set of structural elements that require human intervention to adjust them precisely and in the same way in each case, then the test itself must be performed also with human collaboration, e.g. when the load pressure is exerted.

Another significant disadvantage of the known solutions is that it is almost impossible to perform disc load bearing capacity tests and obtain the same result, due to the structural design and subjective factors. The deviations, i.e. the measurement errors are well known to be significantly caused by the measuring personnel, the speed of applying the load and the resting time between the loading steps, and by the subjective judgement of these conditions.

Our objective with the solution according to the invention was to overcome the deficiencies of the known devices and the assessment procedures performed with them, and to create a simply portable, easily assembled device adapted for the measurement of values that are identical and precise even in the case of repeated measurements that makes it possible to quickly, precisely and reliably determine numerous physical parameters of the tested soil or particulate material without error, which parameters are important from the aspects of civil and construction engineering, railways, road and water facility construction, including static compaction independently of human intervention, on the basis of which during implementation of the engineering project the optimum construction technology may be determined, and so the stability and lifetime of the facility may be improved.

The design of the device according to the invention is based on the consideration that if the deflection occurring as a consequence of the load is measured using an unconventional measuring head and reference piece arrangement, then the errors occurring in the case of conventional measurement methods and devices may be overcome. However, for this a measuring head and reference piece arrangement is required where on measuring the change of distance between the two part-units it is possible to precisely measure the depression of the measuring head compared to the reference piece without human intervention while the reference piece remains immobile.

On the basis of the above series of ideas, the recognition that led to the creation of the device according to the invention was that if the device housing is created in an unconventional way so that, on the one part, the reference piece may be shifted on it, and so, in this way the reference piece itself may rest on the tested soil at a sufficient distance from the measurement location so that the given part of the reference piece in the vicinity of the measuring head does not move during measurement, and, on the other part, a measuring head is positioned in the device housing under the reference piece that is able to measure the difference in distance between measuring head and the reference piece in a contactless way and in such a way that the measuring head that is settling downwards compared to the unmoving reference piece essentially measures the value of its own sinking, while the reference piece is in contact neither with the device housing nor with its immediate vicinity in any way whatsoever, then the measured distance value provides a precise value without human intervention, on the basis of which the static compaction of the tested soil or particulate material, and even numerous other important physical properties may be simply and precisely determined, and so the task may be solved.

It was also a part of the recognition that instead of the conventional pumping, a load-transfer apparatus is used, in the case of which the supply of hydraulic fluid to the hydraulic slave cylinder that actually performs the loading and the extraction of the hydraulic fluid may be performed in a programmed way, at a constant speed, then in the case of appropriately selecting the number and the process of the load cycles, the test may be performed simply, quickly, without human intervention, and numerous physical properties may be determined, and so the criterion of being able to repeat the measurement process and obtain a constant result is fulfilled, i.e. the task may be solved.

The recognition in connection with the creation of the method according to the invention was that the composition of the deflection must be measured when the final loading is applied and not after unloading. The reason for this is that the total deflection at the end of each loading step is the sum total of the plastic, elastic and compaction deflection. Due to this it is preferable to determine plastic deflection from the very last loading of a large number of cycles, and in the knowledge of this it is preferable to determine elastic deflection from the loading of one cycle beforehand. In this way then, if, instead of what is conventionally carried out, several complete but partially different loading cycles are performed in a specific order, during which the loading and unloading is performed under specified conditions, i.e. the loading and unloading rate, and the resting time are maintained in a novel way between given values, during the tests it is possible to determine more precise physical properties and significantly more types of more precise property relating to the tested soil or particulate material, with consideration to which decisions may be made and construction technologies selected during the construction of the engineering facility that have a preferable effect on stability and lifetime, and so the task may be solved.

In accordance with the set objective device according to the invention for the determination of the physical properties of soils and particulate materials, particularly their static compaction—which contains a device housing, a load-transfer apparatus, a reference piece and measuring head, there is a seating for accommodating a part of the reference piece, where the seating is connected to the free opening positioned on the delimiting surface of the device housing, the load-transfer apparatus has a hydraulic slave cylinder and piston that has a medium-transfer pipe end, and the measuring head is located in the seating—is set up in such a way that in addition to the free opening the delimiting surface of the device housing has at least one through-opening, and the through-opening is connected to the seating, and so a reference piece passage channel consisting of the seating, the free opening and the through-opening is formed in the device housing, and with the device in operation the reference piece is inserted through the reference piece passage channel, the first end of the reference piece is supported on the surface of the tested soil or particulate material on the first side of the reference piece passage channel by a leg, while the second end of the reference piece is supported on the surface of the tested soil or particulate material at the second side of the reference piece passage channel by a leg, furthermore, the reference piece is inserted into the reference piece passage channel without contact, the measuring head is secured so it is immobile under the reference piece in the seating, and the measuring head has a contactless distance gauge measuring from the measuring head in the direction towards the lower surface of the reference piece.

A further feature of the device according to the invention may be that the reference piece has a first support at its first end and a second support at its second end, the distance between the first support and the second support is at least 2.4 metres, and with the device in operation the first support and the second support are at a distance of at least 1.2 m from the longitudinal axis of the device housing.

In the case of a possible version of the device, the contactless distance gauge of the measuring head is a laser measure.

In the case of another different embodiment of the invention the measuring head is coupled with a transmitter part-unit, and the transmitter part-unit is connected to a processing unit via an information forwarding channel.

In the case of yet another embodiment of the device, the piston is supplemented with an accessory, furthermore there is a first joint piece located at the free end of the piston, while there is a second joint piece located on the connection surface of the accessory facing the piston that operates together with the first joint piece.

From the point of view of the invention it may be preferable if the material of the device housing and/or the reference piece is high tensile strength, light, fibre-reinforced resin, such as carbon fibre Kevlar.

In the case of another embodiment of the device, the device housing has a base plate, and a tower part protruding from the base plate, and the seating is created in the tower part.

Optionally, the device housing is supplemented with reinforcing ribs inserted between the base plate and the tower part.

In the case of another embodiment of the invention a support shell enclosing a receiving space is formed at the end of the tower part opposite the base plate, and when the device is in operation at least a part of the hydraulic slave cylinder is fitted into the receiving space in such a way so that it may be removed.

In the case of another embodiment of the device, a constant flow rate hydraulic pump is connected to the medium-transfer pipe end of the hydraulic slave cylinder.

In accordance with the set objective method according to the invention for the determination of the physical properties of soils and particulate materials, particularly their static compaction—during which a device is used for the determination of static compaction which has a device housing, a measuring head connected to this, and a reference piece linked to the measuring head, the device housing is placed onto the surface of the soil or particulate material to be tested, the device housing is placed under a load mass known of in itself and having a weight in excess if 10 tonnes, then a load-transfer apparatus is inserted between the device housing and the load mass, following this the load-transfer apparatus is tensioned, and so by routing a part of the weight of the load mass through the device housing to the surface of the tested soil or particulate material load pressure is created, and a surface pressed by the load pressure is created, then after maintaining the given pressure for a resting period, a compacted layer is created, after the resting period has elapsed the load-transfer apparatus is tensioned more to create increased load pressure, which is routed to the compacted layer, then after maintaining the given increased pressure for a resting period, an increased pressure compacted layer is created, the pressure-increasing step is repeated at least four times with increasing load pressure, and so a complete loading cycle is performed, at the end of the complete loading cycle the measuring head is used to determine the value of the distance between the measuring head and the reference piece, then the load pressure is removed, and then the physical properties of the tested soil or particulate material are determined from the value obtained,—based on the principle that in the course of the complete loading cycle the load pressure is increased at an even rate from 0 MPa to at least 0.3 MPa, and between the individual load-increase steps and following the final load-increase step a constant resting time selected between 2 to 15 seconds is maintained, then after the greatest pressure is achieved the pressure is reduced at an even rate to 0 MPa, and the value of the distance between the measuring head and the reference piece is determined using the measuring head, and the deflection value obtained, along with the corresponding pressure value, is recorded, following this a second loading cycle is carried out in at least three steps, in the course of which the load pressure is increased at an even rate from 0 MPa to at least 0.3 MPa, and between the individual load-increase steps and following the final load-increase step a constant resting time selected between 2 to 15 seconds is maintained, then after the greatest pressure is achieved and the resting time has elapsed, the value of the distance between the measuring head and the reference piece is determined using the measuring head, and the deflection value obtained, along with the corresponding pressure value, is recorded, following this the pressure is reduced to 0 MPa at an even rate, and the value of the distance between the measuring head and the reference piece is determined using the measuring head, and the permanent deflection value obtained, along with the corresponding pressure value, is recorded, following this a third loading cycle is carried out in at least two steps, in the course of which the load pressure is increased at an even rate from 0 MPa to at least 0.3 MPa, and between the individual load-increase steps and following the final load-increase step a constant resting time selected between 2 to 15 seconds is maintained, then after the greatest pressure is achieved and the resting time has elapsed, the value of the distance between the measuring head and the reference piece is determined using the measuring head, and the deflection value obtained, along with the corresponding pressure value, is recorded, following this the pressure is reduced to 0 MPa at an even rate, and the value of the distance between the measuring head and the reference piece is determined using the measuring head, and the permanent deflection value obtained, along with the corresponding pressure value, is recorded, following this a fourth loading cycle is carried out in one step, in the course of which the load pressure is increased at an even rate from 0 MPa to at least 0.3 MPa, and following the load increase a resting time selected between 2 to 15 seconds is maintained, then after the resting time has elapsed the value of the distance between the measuring head and the reference piece is determined using the measuring head, and the deflection value obtained, along with the corresponding pressure value, is recorded, following this the pressure is reduced to 0 MPa at an even rate, and the value of the distance between the measuring head and the reference piece is determined using the measuring head, and the permanent deflection value obtained, along with the corresponding pressure value, is recorded, following this a fifth loading cycle is carried out, again in one step, in the course of which the load pressure is increased at an even rate from 0 MPa to at least 0.3 MPa, and following the load increase a resting time selected between 2 to 15 seconds is maintained, then after the resting time has elapsed the value of the distance between the measuring head and the reference piece is determined using the measuring head, and the deflection value obtained, along with the corresponding pressure value, is recorded, then at this time a resting time is observed that is 5 to 15 times the previous values, and the value of the distance between the measuring head and the reference piece is determined once again using the measuring head, and the plastic deflection value obtained, along with the corresponding pressure value, is recorded, following this the pressure is reduced to 0 MPa at an even rate, and the value of the distance between the measuring head and the reference piece is determined using the measuring head, and the permanent deflection value obtained, along with the corresponding pressure value, is recorded, and finally the physical properties of the soil or particulate material, such as the degree of static compaction, are determined from the values obtained.

An additional feature of the method according to the invention may be that during the second loading cycle the load-increase steps are performed as pressure steps equal to twice the value of the first load-increase pressure step, while during the third load-increase the load-increase steps are performed as pressure steps equal to three times the value of the first load-increase pressure step.

In the case of a possible implementation of the method, the magnitude of the unloading duration is selected to be equal to the load-increase time and the resting time between the load-increase steps.

From the point of view of the method it may be preferred if the fourth loading cycle is repeated at least twice, and the fifth loading cycle is performed only following this.

In the case of another different implementation of the method according to the invention when the load-increases are performed, in the case of the load-increase steps the magnitude of the load is increased at a selected, constant rate of between 0.005 to 0.2 MPa/sec, furthermore, when unloading, in the case of the unloading steps the magnitude of the load is reduced as a selected, constant rate of between 0.005 to 0.2 MPa/sec, and the load increasing and unloading rate are selected to be of equal values.

The device according to the invention has numerous preferable characteristics. The most important of these is that as a consequence of the unique device housing provided with the reference piece passage channel, the measuring head located in this at the bottom, and the reference piece secured above the measuring head so it is immobile, distance data may be generated originating from soil depression without human intervention, which excludes the possibility of error, and in this way the device is capable of fulfilling the condition of back-measuring, which could not be performed to date with the known devices.

It may also be viewed as an advantage that the use of the immobile reference piece and the measurement made from a lower point in the upwards direction, the bending or curvature of the reference piece is unable to cause any error during the measurements, only the vibration of the reference piece should be avoided, which, however, may be simply performed through the appropriate selection of the size of the reference piece and the design of the legs.

It must also be viewed as an advantage that the reference piece being inserted through the device housing and supported on the two sides of the housing overcomes the possibility of error that would originate from the deflection trough created due to the loading during the test.

It is a great advantage that no change is required in the structure of the device to determine the degree of static compaction, and due to this the measurement of load capacity and compaction may be performed in a single measurement process, using the same device, and from this measurement both the degree of compaction and the load capacity modulus may be determined.

Another advantage is that due to the use of the processing unit and the programmable constant flow rate hydraulic pump fast and, as required, paperless measurement results may be produced simply, and transmitted to a remote-access information technology centre to be processed.

The advantages originating from the design of the device housing and the load-transfer apparatus, their connection and the selection of the material of the device housing and reference piece include ease of use, quick assembly and disassembly, as well as simple transportation performed with a low volume requirement, which has a preferable effect on the efficiency of the measurements.

An additional advantage of the modular construction is that in the case of a fault in one structural unit, it may be replaced quickly without any particular specialist knowledge being necessary, which further improves the ease of use of the device.

Another advantage of the use of the processing unit and the programmable constant flow rate hydraulic pump is that it is possible to select and adjust the loading and unloading rate and time applied in the method, and the resting time applied in the individual steps and maintain these values at constant levels during the test, which excludes the errors and uncertainties involved, and so may lead to more precise measurement results.

An advantage of the method according to the invention is that the test may be repeated using the device immediately a disc-size further away, as a result of which a good parallel measurement result may be obtained. This may also be carried out in several directions, from which statistic characteristics may be calculated and the homogeneity of the material may also be determined.

An advantage of the method is that purely plastic, purely elastic and purely compaction deflection may be determined from the deflections measured in the course of the five loading cycle. Then in the knowledge of this several types of modulus may be determined in addition to load capacity modulus (resilience modulus and Young's modulus from purely flexible deflection). Furthermore, using known relationships additional data may be calculated from the measured data, such as CBR %, c N/mm3, Evd, Ed, Evib, G, Lat, Lon, S_{25.5} value, SPF (state-parameter factor) and compaction rate, as a result of which the negative risk of decisions may be significantly reduced.

### BRIEF DESCRIPTION OF THE DRAWINGS

The device according to the invention will be explained in more detail by way of exemplary embodiments with reference to figures, wherein

FIG. 1 depicts a side view of the device according to the invention, in partial cross-section

FIG. 2 shows the cross-section of the device according to FIG. 1 taken on the plane II-II.

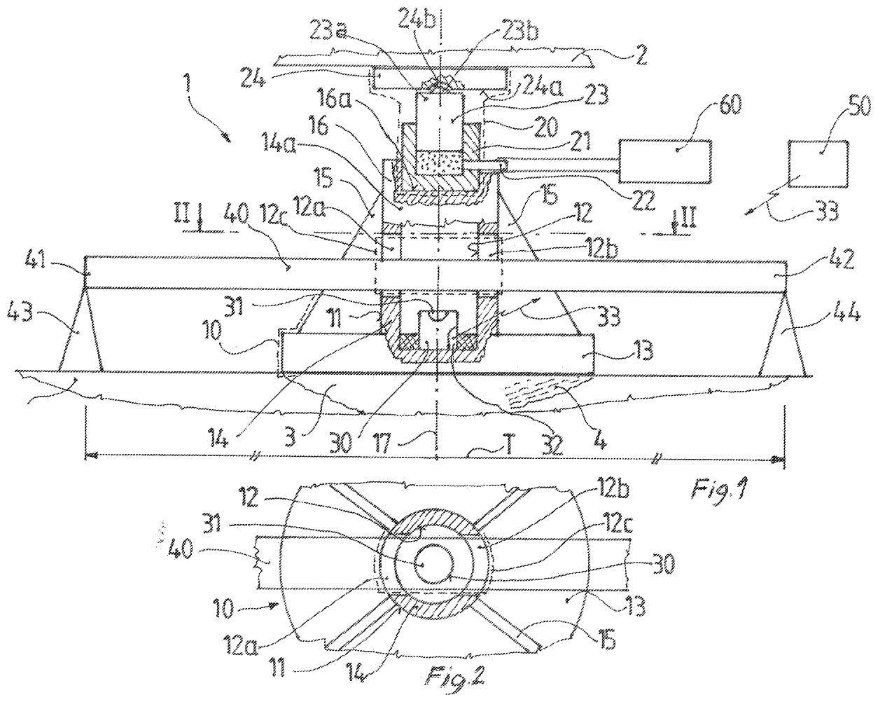

FIG. 1 shows a possible version of the device 1 adapted for determining the static compaction of tested soil or particulate material. It may be observed that in the case of the given embodiment the base plate 13 and the tower part 14 together constitute the device housing 10. In this case the base plate 13 and the tower part 14 are produced from a single piece. In the interest of better load-bearing capacity and more even load transfer, reinforcing ribs 15 are created between the base plate 13 and tower part 14 of the device housing 10. To make operation easier the device housing 10, i.e. the base plate 13, the tower part 14 and the reinforcing ribs 15 are made from carbon fibre Kevlar, but they may also be made of other materials, preferably high tensile strength, light, fibre-reinforced resin.

It is illustrated well in FIG. 1 and particularly well in FIG. 2 that in the case of the present version that a cylindrical ring shaped tower part 14 surrounds the seating 12, and the base plate 13 is a circular disc. It should be noted here, however, that both the tower part 14 and the base plate 13 may be a solid in the shape of a rectangular prism or other shape. It is also well illustrated that the free opening 12a is formed on the first side of the delimiting surface 11 of the device housing 10, here the shell of the tower part 14, and opposite this the through-opening 12b is formed on the other side of the shell, both of which are connected with the seating 12. In this way the free opening 12a, the seating 12 and the through-opening together constitute the reference piece passage channel 12c. This reference piece passage channel 12c serves for guiding through the reference piece 40 used in the measuring process.

It is also preferable for the reference piece 40 to be made from Kevlar or from high tensile strength, light, fibre-reinforced resin. Essentially the reference piece 40 is a 2.4-metre long, preferably rectangular cross-section rod or pipe, with a first support 43 located at its first end 41 and a second support 44 located at its second end 42. For ease of use the first support 43 and the second support 44 may be folded in next to the reference piece 40, and optionally even the reference piece 40 itself may either be folded up or pushed together telescopically. This is important because in this way it is easier to transport, handle and store the reference piece 40 in periods between measurements.

Returning now to FIG. 1, it shows that the measuring head 30 is positioned in the seating 12 of the tower part 14, which measuring head 30 here has a contactless distance gauge 31, preferably a laser measure, and a transmitter part-unit 32 connected to it. Here the transmitter part-unit 32 is connected to the processing unit 50 via the information forwarding channel 33, which processing unit 50 may be a portable computer or even a smartphone. The processing unit 50 has the application that is adapted for receiving, interpreting and evaluating the values measured by the contactless distance gauge 31, as a result of which the physical properties of the tested soil or particulate material, i.e. static compaction that takes into consideration the until now not uniformly defined plastic, elastic and permanent deflection and their proportions, may be determined.

It is important to highlight that the transmitter part-unit 32 may be a wired or wireless version. In this way, such as in the case of RF communication, the processing unit 50 may be even a greater distance from the device 1. In other words, it is possible for the processing unit 50 to be in a remote-access centre, and for the forwarded data to be processed and evaluated there, even in paperless form.

It is also important to mention that the measuring head 30 is inserted in the tower part 14 or the base plate 13 of the device housing 10 in such a way that it may be removed. The reason for this is so that the measuring head 30 may be simply and quickly replaced in the case it becomes faulty.

In the case of the given device 1 there is a support shell 16 at the end 14a of the tower part 14 of the device housing 10, which encloses the receiving space 16a. The hydraulic slave cylinder 21 constituting a part of the load-transfer apparatus 20 may be fitted into the receiving space 16a.

The load-transfer apparatus 20 also includes a piston 23, as well as an accessory 24. These make it possible for a part of the pressure originating from the load mass 2 weighing at least 10 tonnes to be “transferred” to the base plate 13 of the device housing 10, and through this to the tested soil or particulate material.

The appropriate jointed connection of the piston 23 and the accessory 24 is made possible by the first joint piece 23b located at the free end 23a of the piston 23 and the second joint piece 24b formed on the connection surface 24a of the accessory 24 and cooperating with the first joint piece 23b. The design of the first joint piece 23b and the second joint piece 24b is such so that it makes the accessory 24 self-positioning, which in this way is able to rest up against the load mass 2 with its entire surface. It must be mentioned here that the load mass 2 may also be a piece of construction industry heavy-duty machinery, also used during conventional measuring procedures, such as a road roller, a front end loader, tipper truck, etc.

The hydraulic slave cylinder 21 of the load-transfer apparatus 20 has medium-transfer pipe end 22, through which the piston space, which is not indicated in the figures, may be connected to the constant flow rate hydraulic pump 60. The task of the constant flow rate hydraulic pump 60 is to provide a programmed, constant rate of flow of oil during the measuring process during the loading and unloading of the piston. It should be noted here that in the case of the use of the constant flow rate hydraulic pump 60, the processing unit 50 may also control the operation of the constant flow rate hydraulic pump 60.

In FIG. 1 it may be seen that in the given position the compacted layer 3 of the soil or particulate material to be tested is located under the base plate 13 of the device housing 10 of the device 1, which the device 1, while in operation, transforms into an increased load compacted layer 4. This is highlighted by the hatched detail on the right side of FIG. 1.

The assembly of the device 1 according to the invention takes place in the following way. First of all, the measuring head 30 must be fitted into the seating 12 of the device housing 10, then the device housing 10 must be placed onto the tested soil or particulate material so that the base plate 13 is positioned horizontally. Following this the hydraulic slave cylinder 21 of the load-transfer apparatus 20 may be placed into the receiving space 16a of the support shell 16 at the end 14a of the tower part 14 of the device housing 10. After this the medium-transfer pipe end 22 of the hydraulic slave cylinder 21 must be connected to the constant flow rate hydraulic pump 60. Then the reference piece 40 needs to be pushed through the reference piece passage channel 12c of the device housing 10, then by folding down the first support 43 then the second support 44 the reference piece 40 must be placed onto the tested soil or particulate material so that the centre point of the reference piece 40 falls substantially in the longitudinal axis 17 of the device housing 10, in other words, the first support 43 and the second support 44 of the reference piece 40 are positioned symmetrically with respect to the longitudinal axis 17 of the device housing 10 on the tested soil or particulate material. Attention must also be paid to that the reference piece 40 does not come into contact with the surface of the device housing 10 delimiting the reference piece passage channel 12c.

Then as the final step the accessory 24 may be placed onto the free end 23a of the piston 23 of the load-transfer apparatus 20 so that the first joint piece 23b at the free end 23a of the piston 23 and the second joint piece 24b formed on the connection surface 24a of the accessory 24 become coupled.

Then the device 1 is ready for use. After the device 1 has been assembled the heavy-duty machinery constituting the load mass 2 needs to be steered to above the accessory 24 of the device 1, then by starting the constant flow rate hydraulic pump 60 the piston 23 must be brought into a position so that the accessory 24 comes into contact with the surface of the load mass 2. Following this the measuring head 30 may be activated by switching on the processing unit 50, and so the test may be started. During the test in accordance with the program that has been loaded the constant flow rate hydraulic pump 60 either supplies the work medium into the hydraulic slave cylinder 21 of the load-transfer apparatus 20 and with this it forces the piston 23 between the load mass 2 and the device housing 10, or withdraws the work medium from the hydraulic slave cylinder 21 and terminates the tension between the load mass 2 and the device housing 10.

When the piston 23 is tensioned, as the load mass is at least 10 tonnes, the force pushes the base plate 13 of the device housing 10 into the tested soil or particulate material. As a result of this the device housing 10 moves in the direction of the tested soil or particulate material, i.e. the measuring head 30 recedes downwards from the reference piece 40 pushed through the reference piece passage channel 12c of the device housing 10. And as the first support 43 and the second support 44 of the reference piece 40 are sufficiently distant from the deflection zone, the reference piece 40 remains motionless in its position. As a result of this arrangement, this downwards displacement is measured by the contactless distance gauge 31 of the measuring head 30, which here is a laser measure. Then the given value is transmitted by the transmitter part-unit 32 through the information forwarding channel 33 to the processing unit 50.

As a consequence of the pressure exerted on the tested soil or particulate material a compacted layer 3 is formed in the area under the base plate 13 of the device housing 10.

When the time specified in the measuring program has elapsed and the pressure exerted on the piston 23 drops, because the constant flow rate hydraulic pump 60 is disengaged, the tested soil or particulate material that had been maintained under pressure until then rises back a little. At this time the distance between the contactless distance gauge 31 of the measuring head 30 and the reference piece 40 is also reduced. This change is also measured by the measuring head 30, and the transmitter part-unit 32 transmits this value via the information forwarding channel 33 to the processing unit 50, which value may be coupled with the also continuously measured value of the current pressure.

During the next loading cycle, the base plate 13 of the device housing 10 is depressed more into the tested soil or particulate material than previously, and in this way an increased load compacted layer 4 is created. The loading cycles consisting of loading and unloading steps succeed each other in accordance with the program. And after the entire measurement has been run the processing unit 50 evaluates the data received and, using the program running in it, determines the physical parameters characteristic of the tested soil or particulate material, including the value of static compaction.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding Hungarian application No. P2300018, filed Jan. 9, 2023, are incorporated by reference herein.

In the following details of the method according to the invention will be presented in connection with examples.

### EXAMPLE 1

In the given method example a soil test in advance of constructing the foundations of a building was performed using the device 1 according to the invention, in the course of which the degree of static compaction was also determined as follows. The device 1 assembled according to that disclosed previously and placed into operation at the site of the measurement was switched on, and with this the measuring program loaded onto the processing unit 50 was launched. During the measurement five full loading cycles were performed.

In the course of the first loading cycle the pressure exerted on the piston 23 was increased in six steps by 0.05 MPa per step from 0.00 MPa to 0.30 MPa, in such a way that in each step the load was increased evenly over 5 seconds, i.e. the load was increased by 0.01 MPa per second.

After the first loading step, i.e. after reaching a pressure of 0.05 MPa, the pressure achieved was maintained for a period of between 2 to 15 seconds, in this case in all cases for 5 seconds. At the end of the resting period due to the effect of the pressure the tested soil had become compressed, deflection had taken place, and in this way a first compacted layer 3 was formed under the base plate 13 of the device housing 10. After the resting period had expired the distance between the contactless distance gauge 31 of the measuring head 30 and the reference piece 40 was determined with a precision of 0.01 mm or 0.001 mm, and the value was transmitted to the processing unit 50 by the transmitter part-unit 32 via the information forwarding channel 33, and with this the current pressure value produced by the constant flow rate hydraulic pump 60 corresponding to this deflection value was forwarded to the processing unit 50.

In the second loading step the pressure was increased from 0.05 MPa to 0.1 MPa, and once again it was maintained for a resting period of 5 seconds on the tested soil. Then in this way after the first loading step an increased load compacted layer 4 was created under the vase disc 13 of the device housing 10. After the resting period has expired the distance between the contactless distance gauge 31 of the measuring head 30 and the reference piece 40 was determined with a precision of 0.01 mm or 0.001 mm, and the value was transmitted to the processing unit 50 with the transmitter part-unit 32 via the information forwarding channel 33, and with this the current pressure value of the constant flow rate hydraulic pump 60 was forwarded to the processing unit 50.

Then the loading steps were performed in the same way up until the sixth loading step. After the sixth loading step, when the load was increased from 0.25 MPa to 0.30 MPa, and the distance between the contactless distance gauge 31 of the measuring head 30 and the reference piece 40 was determined with a precision of 0.01 mm or 0.001 mm, and then forwarded, the pressure exerted on the piston 23 was reduced in one step even over the course of 60 seconds from 0.30 MPa to 0.00 MPa, i.e. the load-transfer apparatus 20 was unloaded. Then the distance between the contactless distance gauge 31 of the measuring head 30 and the reference piece 40 was determined with a precision of 0.01 mm or 0.001 mm, and the value was transmitted to the processing unit 50 by the transmitter part-unit 32 via the information forwarding channel 33. At the end of this first loading cycle the deflection of the tested soil was recorded along with the current pressure values also forwarded, and with this the first full loading cycle was completed.

Immediately after the first loading cycle the second complete loading cycle was performed as follows. In the course of the second full loading cycle the pressure exerted on the piston 23 was increased in three steps by 0.01 MPa per step from 0.00 MPa to 0.30 MPa, in such a way that in each step the load was increased evenly over 10 seconds, i.e. the load was increased by 0.01 MPa per second.

After the individual loading steps were performed, the pressure exerted was maintained for a period of between 2 and 15 seconds, in this case in all cases for 5 seconds. After the resting periods had expired the distance between the contactless distance gauge 31 of the measuring head 30 and the reference piece 40 was determined with a precision of 0.01 mm or 0.001 mm, and the values were transmitted to the processing unit 50 by the transmitter part-unit 32 via the information forwarding channel 33, where the pressure values corresponding to the given values were also forwarded.

After the third loading step, when the load was increased from 0.20 MPa to 0.30 MPa, the distance between the contactless distance gauge 31 of the measuring head 30 and the reference piece 40 was determined with a precision of 0.01 mm or 0.001 mm. The given deflection value was transmitted to the processing unit 50 by the transmitter part-unit 32 via the information forwarding channel 33, as was the pressure value corresponding to the given deflection value, and then the data were stored.

Then following the third loading step, when the load was increased from 0.20 MPa to 0.30 MPa, and the distance between the contactless distance gauge 31 of the measuring head 30 and the reference piece 40 was determined with a precision of 0.01 mm or 0.001 mm, and forwarded, the pressure exerted on the piston 23 was reduced in one step, evenly over 45 seconds from 0.30 MPa to 0.00 MPa, i.e. the load-transfer apparatus 20 was unloaded. Then the distance between the contactless distance gauge 31 of the measuring head 30 and the reference piece 40 was determined with a precision of 0.01 mm or 0.001 mm, and the value was transmitted to the processing unit 50 by the transmitter part-unit 32 via the information forwarding channel 33, where the pressure value corresponding to the given distance value was also sent. With this, at the end of the second complete loading cycle, after the unloading the given permanent deflection of the tested soil was also recorded.

After the second loading cycle, the consecutive third complete loading cycle was performed as follows. In the course of the third full loading cycle the pressure exerted on the piston 23 was increased in two steps by 0.15 MPa per step from 0.00 MPa to 0.30 MPa, in such a way that in each step the load was increased evenly over 15 seconds, i.e. the load was increased by 0.01 MPa per second.

After the first loading step was performed, the pressure exerted was maintained for a period of between 2 and 15 seconds, in this case in all cases for 5 seconds. After the resting periods had expired the distance between the contactless distance gauge 31 of the measuring head 30 and the reference piece 40 was determined with a precision of 0.01 mm or 0.001 mm, and the values were transmitted to the processing unit 50 by the transmitter part-unit 32 via the information forwarding channel 33, and then stored with the current pressure values. After the unloading following the first loading step, the permanent deflection was also recorded.

After the second loading step, when the load was increased from 0.15 MPa to 0.30 MPa, the distance between the contactless distance gauge 31 of the measuring head 30 and the reference piece 40 was determined with a precision of 0.01 mm or 0.001 mm. The given deflection value was transmitted to the processing unit 50 by the transmitter part-unit 32 via the information forwarding channel 33, and was stored along with the current pressure value. After the unloading following the second loading step, the permanent deflection was also recorded.

Then following the second loading step, when the load was increased from 0.15 MPa to 0.30 MPa, and the distance between the contactless distance gauge 31 of the measuring head 30 and the reference piece 40 was determined with a precision of 0.01 mm or 0.001 mm, and forwarded, the pressure exerted on the piston 23 was reduced in one step, evenly over 40 seconds from 0.30 MPa to 0.00 MPa, i.e. the load-transfer apparatus 20 was unloaded. Then the distance between the contactless distance gauge 31 of the measuring head 30 and the reference piece 40 was determined with a precision of 0.01 mm or 0.001 mm, and the value was transmitted to the processing unit 50 by the transmitter part-unit 32 via the information forwarding channel 33, along with the current pressure value. With this, at the end of the third complete loading cycle, after the unloading the given permanent deflection of the tested soil was also recorded.

After the third loading cycle, the consecutive fourth complete loading cycle was performed as follows. In the course of the fourth full loading cycle the pressure exerted on the piston 23 was increased in a single step from 0.00 MPa to 0.30 MPa evenly over 30 seconds, i.e. here too the load was increased by 0.01 MPa per second.

After the loading step was performed, the pressure exerted was maintained for a period of between 2 and 15 seconds, in this case too for 5 seconds. After the resting period had expired the distance between the contactless distance gauge 31 of the measuring head 30 and the reference piece 40 was determined with a precision of 0.01 mm or 0.001 mm, and the deflection values obtained were transmitted to the processing unit 50 by the transmitter part-unit 32 via the information forwarding channel 33, and then stored with the current pressure values.

Then the pressure exerted on the piston 23 was reduced in one step, evenly over 35 seconds from 0.30 MPa to 0.00 MPa, i.e. the load-transfer apparatus 20 was unloaded. Then the distance between the contactless distance gauge 31 of the measuring head 30 and the reference piece 40 was determined with a precision of 0.01 mm or 0.001 mm, and the permanent deflection value obtained was transmitted to the processing unit 50 by the transmitter part-unit 32 via the information forwarding channel 33, and recorded, furthermore the permanent deflection value was also stored.

After the fourth full loading cycle, in the case of the given version of the method, a final, fifth full loading cycle was performed. In this full loading cycle the pressure exerted on the piston 23 was also increased in a single step from 0.00 MPa to 0.30 MPa evenly over 30 seconds, i.e. here the load was increased by 0.01 MPa per second.

After the loading step was performed, the pressure exerted was maintained for a period of between 2 and 15 seconds, in this case too for 5 seconds. After the resting period had expired the distance between the contactless distance gauge 31 of the measuring head 30 and the reference piece 40 was determined with a precision of 0.01 mm or 0.001 mm, and the deflection values obtained were transmitted to the processing unit 50 by the transmitter part-unit 32 via the information forwarding channel 33, and then stored with the current pressure values.

However, in the given full loading cycle, after this the pressure level was maintained for a duration of 5 to 15 times the previous resting period, in this case for 25 seconds, then the distance between the contactless distance gauge 31 of the measuring head 30 and the reference piece 40 was determined with a precision of 0.01 mm or 0.001 mm, and the obtained values corresponding to plastic deflection were transmitted to the processing unit 50 by the transmitter part-unit 32 via the information forwarding channel 33 along with the current pressure values, and then stored.

Then the pressure exerted on the piston 23 was reduced in one step, evenly over 30 seconds from 0.30 MPa to 0.00 MPa, i.e. the load-transfer apparatus 20 was unloaded. Then the distance between the contactless distance gauge 31 of the measuring head 30 and the reference piece 40 was determined with a precision of 0.01 mm or 0.001 mm, and the permanent deflection value obtained was transmitted to the processing unit 50 by the transmitter part-unit 32 via the information forwarding channel 33, and recorded.

The physical features of the tested soil, i.e. the static compaction value, and in detail the plastic and elastic and compaction deflection values and other types of modulus were determined from the deflection and pressure data collected in the course of the five full loading cycles and using the program running in the processing unit 50.

### EXAMPLE 2

In the case of the given version of the method, the process disclosed in example 1 was followed identically with the difference that the test was performed 30 to 50 cm away from the previous location, but with the same device 1. As the measurement was performed automatically, without human intervention, the parallel measurement results were produced in 6 minutes, which were compared with the results of the previous test. In this way by calculating the average of the measurement values characteristic of the soil and its conditions, we obtained an even more precise result.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

List of reference signs

```
  1 device
  2 load mass
  3 compacted layer
  4 increased load compacted layer
```

| | |
|---|---|
| 10 device housing | 11 delimiting surface |
| | 12 seating |
| | 12a free opening |
| | 12b through-opening |
| | 12c reference piece passage channel |
| | 13 base plate |
| | 14 tower part |
| | 14a end |
| | 15 reinforcing ribs |
| | 16 support shell |
| | 16a receiving space |
| | 17 longitudinal axis |
| 20 load-transfer apparatus | 21 hydraulic slave cylinder |
| | 22 medium-transfer pipe end |
| | 23 piston |
| | 23a free end |
| | 23b first joint piece |
| | 24 accessory |
| | 24a connection surface |
| | 24b second joint piece |
| 30 measuring head | 31 contactless distance gauge |
| | 32 transmitter part-unit |
| | 33 information forwarding channel |
| 40 reference piece | 41 first end |
| | 42 second end |
| | 43 first support |
| | 44 second support |

50 processing unit
60 constant flow rate hydraulic pump
T distance

1. Device for the determination of the physical properties of soils and particulate materials, particularly their static compaction, which contains a device housing (10), a load-transfer apparatus (20), a reference piece (40) and measuring head (30), there is a seating (12) for accommodating a part of the reference piece (40), where the seating (12) is connected to the free opening (12a) positioned on the delimiting surface (11) of the device housing (10), the load-transfer apparatus (20) has a hydraulic slave cylinder (21) and piston (23) that has a medium-transfer pipe end (22), and the measuring head (30) is located in the seating (12), characterised by that in addition to the free opening (12a) the delimiting surface (11) of the device housing (10) has at least one through-opening (12b), and the through-opening (12b) is connected to the seating (12), and so a reference piece passage channel (12c) consisting of the seating (12), the free opening (12a) and the through-opening (12b) is formed in the device housing (10), and with the device (1) in operation the reference piece (40) is inserted through the reference piece passage channel (12c), the first end (41) of the reference piece (40) is supported on the surface of the tested soil or particulate material on the first side of the reference piece passage channel (12c), while the second end (42) of the reference piece (40) is supported on the surface of the tested soil or particulate material at the second side of the reference piece passage channel (12c), furthermore, the reference piece (40) is inserted into the reference piece passage channel (12c) without contact, the measuring head (30) is secured so it is immobile under the reference piece (40) in the seating (12), and the measuring head (30) has a contactless distance gauge (31) measuring from the measuring head (30) in the direction towards the lower surface of the reference piece (40).

2. Device according to claim 1, characterised by that the reference piece (40) has a first support (43) at its first end (41) and a second support (44) at its second end (42), the distance (T) between the first support (43) and the second support (44) is at least 2.4 metres, and with the device in operation the first support (43) and the second support (44) are at a distance of at least 1.2 m from the longitudinal axis (17) of the device housing (10).

3. Device according to claim 1, characterised by that the contactless distance gauge (31) of the measuring head (30) is a laser measure.

4. Device according to claim 1, characterised by that the measuring head (30) is coupled with a transmitter part-unit (32), and the transmitter part-unit (32) is connected to a processing unit (50) via an information forwarding channel (33).

5. Device according to claim 1, characterised by that the piston (23) is supplemented with an accessory (24), furthermore there is a first joint piece (23b) located at the free end (23a) of the piston (23), while there is a second joint piece (24b) located on the connection surface (24a) of the accessory (24) facing the piston (23) that operates together with the first joint piece (23b).

6. Device according to claim 1, characterised by that the material of the device housing (10) and/or the reference piece (40) is high tensile strength, light, fibre-reinforced resin, such as carbon fibre Kevlar.

7. Device according to claim 1, characterised by that the device housing (10) has a base plate (13), and a tower part (14) protruding from the base plate (13), and the seating (12) is created in the tower part (14).

8. Device according to claim 7, characterised by that the device housing (10) is supplemented with reinforcing ribs (15) inserted between the base plate (13) and the towerpart (14).

9. Device according to claim 7, characterised by that a support shell (16) enclosing a receiving space (16a) is formed at the end (14a) of the tower part (14) opposite the base plate (13), and when the device (1) is in operation at least a part of the hydraulic slave cylinder (21) is fitted into the receiving space (16a) in such a way so that it may be removed.

10. Device according to claim 1, characterised by that a constant flow rate hydraulic pump (60) is connected to the medium-transfer pipe end (22) of the hydraulic slave cylinder (21).

11. Method for the determination of the physical properties of soils and particulate materials, particularly their static compaction, during which a device (1) is used for the determination of static compaction which has a device housing (10), a measuring head (30) connected to this, and a reference piece (40) linked to the measuring head (30), the device housing (10) is placed onto the surface of the soil or particulate material to be tested, the device housing (10) is placed under a load mass (2) known of in itself and having a weight in excess if 10 tonnes, then a load-transfer apparatus (20) is inserted between the device housing (10) and the load mass (2), following this the load-transfer apparatus (20) is tensioned, and so by routing a part of the weight of the load mass (2) through the device housing (10) to the surface of the tested soil or particulate material load pressure is created, and a surface pressed by the load pressure is created, then after maintaining the given pressure for a resting period, a compacted layer (3) is created, after the resting period has elapsed the load-transfer apparatus (20) is tensioned more to create increased load pressure, which is routed to the compacted layer (3), then after maintaining the given increased pressure for a resting period, an increased pressure compacted layer (4) is created, the pressure-increasing step is repeated at least four times with increasing load pressure, and so a complete loading cycle is performed, at the end of the complete loading cycle the measuring head (30) is used to determine the value of the distance between the measuring head (30) and the reference piece (40), then the load pressure is removed, and then the physical properties of the tested soil or particulate material are determined from the value obtained, characterised by that in the course of the complete loading cycle the load pressure is increased at an even rate from 0 MPa to at least 0.3 MPa, and between the individual load-increase steps and following the final load-increase step a constant resting time selected between 2 to 15 seconds is maintained, then after the greatest pressure is achieved the pressure is reduced at an even rate to 0 MPa, and the value of the distance between the measuring head (30) and the reference piece (40) is determined using the measuring head (30), and the deflection value obtained, along with the corresponding pressure value, is recorded, following this a second loading cycle is carried out in at least three steps, in the course of which the load pressure is increased at an even rate from 0 MPa to at least 0.3 MPa, and between the individual load-increase steps and following the final load-increase step a constant resting time selected between 2 to 15 seconds is maintained, then after the greatest pressure is achieved and the resting time has elapsed, the value of the distance between the measuring head (30) and the reference piece (40) is determined using the measuring head (30), and the deflection value obtained, along with the corresponding pressure value, is recorded, following this the pressure is reduced to 0 MPa at an even rate, and the value of the distance between the measuring head (30) and the reference piece (40) is determined using the measuring head (30), and the permanent deflection value obtained, along with the corresponding pressure value, is recorded, following this a third loading cycle is carried out in at least two steps, in the course of which the load pressure is increased at an even rate from 0 MPa to at least 0.3 MPa, and between the individual load-increase steps and following the final load-increase step a constant resting time selected between 2 to 15 seconds is maintained, then after the greatest pressure is achieved and the resting time has elapsed, the value of the distance between the measuring head (30) and the reference piece (40) is determined using the measuring head (30), and the deflection value obtained, along with the corresponding pressure value, is recorded, following this the pressure is reduced to 0 MPa at an even rate, and the value of the distance between the measuring head (30) and the reference piece (40) is determined using the measuring head (30), and the permanent deflection value obtained, along with the corresponding pressure value, is recorded, following this a fourth loading cycle is carried out in one step, in the course of which the load pressure is increased at an even rate from 0 MPa to at least 0.3 MPa, and following the load increase a resting time selected between 2 to 15 seconds is maintained, then after the resting time has elapsed the value of the distance between the measuring head (30) and the reference piece (40) is determined using the measuring head (30), and the deflection value obtained, along with the corresponding pressure value, is recorded, following this the pressure is reduced to 0 MPa at an even rate, and the value of the distance between the measuring head (30) and the reference piece (40) is determined using the measuring head (40), and the permanent deflection value obtained, along with the corresponding pressure value, is recorded, following this a fifth loading cycle is carried out, again in one step, in the course of which the load pressure is increased at an even rate from 0 MPa to at least 0.3 MPa, and following the load increase a resting time selected between 2 to 15 seconds is maintained, then after the resting time has elapsed the value of the distance between the measuring head (30) and the reference piece (40) is determined using the measuring head (30), and the deflection value obtained, along with the corresponding pressure value, is recorded, then at this time a resting time is observed that is 5 to 15 times the previous values, and the value of the distance between the measuring head (30) and the reference piece (40) is determined once again using the measuring head (30), and the plastic deflection value obtained, along with the corresponding pressure value, is recorded, following this the pressure is reduced to 0 MPa at an even rate, and the value of the distance between the measuring head (30) and the reference piece (40) is determined using the measuring head (30), and the permanent deflection value obtained, along with the corresponding pressure value, is recorded, and finally the physical properties of the soil or particulate material, such as the degree of static compaction, are determined from the values obtained.

12. Method according to claim 11, characterised by that during the second loading cycle the load-increase steps are performed as pressure steps equal to twice the value of the first load-increase pressure step, while during the third load-increase the load-increase steps are performed as pressure steps equal to three times the value of the first load-increase pressure step.

13. Method according to claim 11, characterised by that the magnitude of the unloading duration is selected to be equal to the load-increase time and the resting time between the load-increase steps.

14. Method according to claim 11, characterised by that the fourth loading cycle is repeated at least twice, and the fifth loading cycle is performed only following this.

15. Method according to claim 11, characterised by that when the load-increases are performed, in the case of the load-increase steps the magnitude of the load is increased at a selected, constant rate of between 0.005 to 0.2 MPa/sec, furthermore, when unloading, in the case of the unloading steps the magnitude of the load is reduced as a selected, constant rate of between 0.005 to 0.2 MPa/sec, and the load increasing and unloading rate are selected to be of equal values.
